(19) United States
(12) Patent Application Publication
(10) Pub. No.: US 2007/0244444 A1
(43) Pub. Date: Oct. 18, 2007

(54) Adjustable dosage syringe

(76) Inventors: Benjamin Richard Guelker, Duluth, GA (US); Patrick Wayne Thompson, Greenville, SC (US)

Correspondence Address: Judy Jarecki-Black,Ph.D.,J.D, 3239 Satellite Blvd., Duluth, GA, 30096, US

(21) Appl. No.: 11/716,825

(22) Filed: Mar. 12, 2007

Related U.S. Application Data
(60) Provisional application No. 60/781,831, filed on Mar. 13, 2006.

(51) Int. Cl. A61M 5/00

(52) U.S. Cl. 604/207

(57) ABSTRACT

The present invention is directed to determining, without repeated calculation by an operator, the volume of a non-solid composition remaining in the barrel of a syringe after discharge of a dose of the composition. The present invention provides a syringe barrel, and a plunger having a dose volume scale disposed along the longitudinal axis and orientated such that the numeric values increase in the direction from the proximal end of the plunger towards the discharge end. The dosage syringe also comprises an adjustable dose selector that may be moved to a volume mark of the scale corresponding to a desired dose to be delivered. The dose syringe barrel further comprises an indicator means that may be an opening in the barrel directly above the dose volume scale such that, when the plunger is in a fully retracted position, the window shows the volume of the full capacity of the syringe barrel and when the plunger is depressed, will show the remaining volume in the syringe.

### RELATED PATENTS AND INCORPORATION BY REFERENCE

Each of the patents cited in this text, as well as each document or reference cited in each of the patents are hereby expressly incorporated herein by reference.

### FIELD OF THE INVENTION

The present invention relates to adjustable dose syringes that automatically indicate the volume of dischargeable composition in the barrel of the syringe. The present invention further relates to kits comprising an adjustable syringe and compositions for delivery to a recipient animal.

### BACKGROUND OF THE INVENTION

Adjustable dose syringes have been used for delivering fluid dose to an animal either as a single volume charge of the syringe barrel or as multiple doses from a larger barrel charge. Two alternatives present themselves to the veterinarian or technician. Single dose disposable syringes must be selected to deliver the required dose for a particular animal weight, necessitating that the operator have on hand a large number of different size syringes appropriate for the size distribution of the group of animals to be treated. Disposable syringes are expensive but reduce the likelihood of an error in dosage for a given animal.

Adjustable multiple dose syringes when used to dose multiple animals such as in an agricultural setting, require that the delivered volume be reset for each animal of differing weight, that this be done quickly, and lead to few, if any, errors. Traditional pre-filled oral dosing devices or syringes, used for administering pharmaceuticals to an animal, although capable of being adjusted to deliver a preset volume of the medication, do not inform the operator of the volume of the preparation that might remain in the device. In some cases it is advantageous to use a syringe with a medication volume sufficient for doses to be delivered to more than one animal. It is useful, therefore, for the operator of the device to be aware of the remaining volume of medication after each delivery so that an animal is not inadvertently under dosed. By avoiding the need to calculate the remaining volume available for delivery, errors and time-wasting would be minimized.

There is, therefore, a need for a medication delivery device such as a syringe that is able to accurately deliver multiple doses of a composition while indicating to the operator the volume of the composition remaining in the syringe, thereby avoiding the risk of delivering too small a dose due to inadequate reserve fluid. Whereas adjustable syringes known in the art provide a means to adjust the volume of the composition delivered by a single depression of a plunger, they do not inform the operator of the remaining volume of composition in the barrel of the syringe. Thus, when an operator is administering repeat doses to a series of animals, or needs to vary the dose size in accordance with the weight, etc criteria of each animal, it is necessary to make a calculation to ensure that there is enough composition remaining for the desired dose. This step slows the efficiency when dosing large numbers of animals and may lead to calculation errors and the wrong dose delivered or exhaustion of the supply of composition in the syringe while in dosing a particular animal.

### SUMMARY OF THE INVENTION

Citation or identification of any document in this application is not an admission that such document is available as prior art to the present invention.

The present invention is directed to addressing the problem of determining, without repeated calculation by an operator, the volume of a non-solid composition remaining in the barrel of a syringe after discharge of a dose of the composition. The present invention provides a syringe barrel and a plunger, the plunger having a dose volume scale disposed along the longitudinal axis and orientated such that the numeric values increase in the direction from the proximal end of the plunger towards the distal end. The dose volume scale may be printed, engraved or otherwise directly marked on the surface of the plunger or the scale may be a strip of such as metal or plastic that is attached along its entire length to the plunger or more loosely by attaching the proximal end, bearing the lowest indicated volume, to the proximal end of the plunger. The dose volume scale may be in any desired units such as, but not limited to lbs or kg of the recipient animal, volume of composition (ml's, cc's etc), units of a pharmaceutical agent and the like.

The dosage syringe of the present invention also comprises an adjustable dose selector disposed at the proximal plunger receiving end of the barrel and configured that it may engage the proximal end of the plunger to prevent movement of the plunger towards the distal discharge end of the barrel that would otherwise deliver a greater volume of the composition than is desired. The dose selector may be moved to a volume mark of the scale corresponding to a desired dose to be delivered. The plunger may then be depressed until it encounters the dose selector, whereupon further depression is resisted.

The barrel of the dose syringe of the invention further comprises an indicator means to indicate from the dose volume scale the amount of non-solid composition in the internal reservoir. In one embodiment of the syringe of the invention, the indicator means may be an opening in the barrel and positioned over the longitudinal axis of the plunger and directly above the dose volume scale such that, when the plunger is in a fully retracted position, the window shows the volume of the full capacity of the syringe barrel. Accordingly, the remaining volume of composition is immediately presented without calculation by the syringe operator.

One aspect of the present invention, therefore, provides a dosage syringe comprising: a syringe barrel; a plunger movably disposed within the barrel and having a length greater than the internal length of the barrel, and having a proximal end configured to transfer an operator hand pressure to the plunger, a distal end having a seal means disposed across the contents of the barrel to prevent the contents from leaking and to transfer the hand pressure to the contents of the barrel for discharging the contents through the distal discharge end, and a dose volume scale disposed along the longitudinal axis of the plunger and orientated such that the numeric value increases in a direction from the proximal end of the plunger towards the distal end of the syringe; an adjustable dose selector disposed at the proximal plunger receiving end of the barrel and configured to engage the proximal end of the plunger when the plunger is depressed, wherein the dose selector cooperates with the dose volume scale of the plunger to indicate a desired dose of a pharmaceutical composition to be delivered by the syringe; and an indicator means disposed on the barrel to indicate on the dose volume scale the amount of the pharmaceutical composition in the internal reservoir.

In the various embodiments of this aspect of the invention, the discharge end of the barrel may be configured for insertion into an orifice of an animal.

One embodiment according to the present invention further comprises a gripping means for securing the syringe during operation.

In another embodiment of the invention, the dosage syringe further comprises an extension to the proximal plunger receiving end of the barrel and having the adjustable dose selector disposed thereon.

In the various embodiments of the invention, the dosage syringe may further comprise at least one protuberance configured to limit the entry of the barrel into the mouth of an animal.

In the embodiments of the invention, the dose volume scale may be engraved, printed or attached along the longitudinal axis of the plunger.

In one embodiment of the invention, the dose volume scale is a linear strip and attached by a proximal end to the proximal end of the plunger and the distal end of the strip is movably disposed along the longitudinal axis of the barrel of the syringe.

In the various embodiments of the dosage syringe according to the invention, the indicator means disposed on the barrel to indicate on the amount of the pharmaceutical composition in the internal reservoir is selected from a window, an indicator mark or an elongated cutout from the barrel. In one embodiment of the invention, the indicator means is a window.

The present invention may further encompass a dosage syringe comprising: a syringe barrel, a plunger movably disposed within the barrel and having a length greater than the internal length of the barrel, and having a proximal end configured to transfer an operator hand pressure to the plunger, a distal end having a seal means disposed across the contents of the barrel to prevent the contents from leaking and to transfer the hand pressure to the contents of the barrel for discharge. A dose volume scale may be disposed along the longitudinal axis of the plunger and orientated such that the numeric value increases in a direction from the proximal end of the plunger, wherein the dose volume scale is engraved, printed, attached along the longitudinal axis of the plunger. Alternatively, the dose scale may be a linear strip attached by a proximal end to the proximal end of the plunger. The distal end of the strip would be movably disposed along the longitudinal axis of the barrel of the syringe. An adjustable dose selector is disposed at the proximal plunger receiving end of the syringe barrel and configured to engage the proximal end of the plunger when the plunger is depressed. The dose selector cooperates with the dose volume scale of the plunger to indicate a desired dose of a pharmaceutical composition to be delivered by the syringe; an indicator means disposed on the barrel to indicate on the dose volume scale the amount of the pharmaceutical composition in the internal reservoir; a gripping means for securing the syringe during operation; an extension to the proximal plunger receiving end of the barrel and having the adjustable dose selector disposed thereon; and at least one protuberance configured to limit the entry of the barrel into the mouth of an animal.

In embodiments of this aspect of the invention, the indicator means disposed on the barrel to indicate on the amount of the non-solid composition in the internal reservoir may be selected from, but not limited to, a window, an indicator mark or an elongated cutout from the barrel.

In one embodiment of this aspect of the invention, the indicator means disposed on the barrel to indicate on the amount of the non-solid composition in the internal reservoir is a window.

Another aspect of the invention provides a kit comprising a dosage syringe according to the present invention, and instructions for the use thereof to deliver a non-solid composition to an animal.

In one embodiment of this aspect of the present invention, the kit further comprises a container enclosing a non-solid composition for delivery to an animal, wherein the non-solid composition may be a fluid or a paste. In another embodiment of the kits of the present invention, the dosage syringe is pre-filled with a non-solid composition for delivery to an animal.

Although methods and materials similar or equivalent to those described herein can be used in the practice or testing of the present invention, suitable methods and materials are described herein. All publications, patent applications, patents, and other references mentioned herein are incorporated by reference in their entirety. In addition, the materials, methods, and examples are illustrative only and are not intended to be limiting.

These and other embodiments are disclosed or are obvious from and encompassed by, the following Detailed Description.

### BRIEF DESCRIPTION OF THE FIGURES

The following detailed description, given by way of example, but not intended to limit the invention solely to the specific embodiments described, may best be understood in conjunction with the accompanying drawings, in which:

FIG. 1 illustrates an embodiment of the dose syringe according to the invention, wherein the syringe barrel has an elongated grip protuberance to provide a counter-force against that pushing the plunger into the barrel, wherein the protuberance and the proximal end of the plunger are configured to be gripped and squeezed together to provide a plunger depressing force.

FIG. 2A illustrates a schematic vertical cross-section of a dose syringe according to the invention, wherein the plunger is in a fully retracted position and the dose volume scale is a strip attached to the plunger. The block arrow indicates the direction of travel possible for the plunger. The arrows ‘a’ and ‘b’ indicate the dose volume scale positions of the maximum capacity of the syringe barrel and the zero mark respectively.

FIG. 2B illustrates a schematic vertical cross-section of a dose syringe according to the invention, wherein the plunger is in a partially depressed position to dead end against the dose selector set at a desired dose volume.

FIG. 2C illustrates a schematic vertical cross-section of a dose syringe according to the invention, wherein the plunger is in a fully depressed position.

FIG. 3A illustrates a top view of an embodiment of a dose syringe according to the invention, wherein the plunger is in a fully retracted position but the dose selector has been set for the syringe to deliver 300 units once the plunger is depressed, and the dose volume scale indicates that there are 1250 units remaining in the barrel.

FIG. 3B illustrates a top view of an embodiment of a dose syringe according to the invention, wherein the plunger is depressed to dead against the dose selector that had been set for the syringe to deliver 300 units, and the dose volume scale indicates that there are 950 units remaining in the barrel.

FIG. 4A illustrates an embodiment of the dose syringe according to the invention, wherein the barrel is elongated to carry the dose selector and the barrel has a protuberance to provide a counter-force against that pushing the plunger into the barrel, wherein the protuberance and the proximal end of the plunger are configured to receive the finger and thumb of an operator.

FIG. 4B illustrates another embodiment of the dose syringe according to the invention, wherein the barrel has an encircling protuberance sized to limit entry of the syringe into an orifice of an animal.

FIG. 4C illustrates another embodiment of the dose syringe according to the invention, wherein the barrel has a cone protuberance to limit entry of the syringe into an orifice of an animal.

FIG. 4D illustrates another embodiment of the dose syringe according to the invention, wherein the barrel has an extension for entering the orifice of an animal and entry therein is limited by an increase in the diameter of the barrel.

FIGS. 5-19 illustrate an embodiment of the dose syringe according to the invention wherein the volume of composition remaining in the barrel of the syringe is indicated from a second volume scale distal to, and in reverse orientation relative to, the dose volume scale.

### DETAILED DESCRIPTION OF THE INVENTION

A full and enabling disclosure of the present invention, including the best mode known to the inventor of carrying out the invention is set forth more particularly in the remainder of the specification, including reference to the accompanying drawings, wherein like reference numerals designate corresponding parts throughout several figures. This description is made for the purpose of illustrating the general principles of the invention and should not be taken in the limiting sense.

As used herein, the following terms have the meanings ascribed to them unless specified otherwise. In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

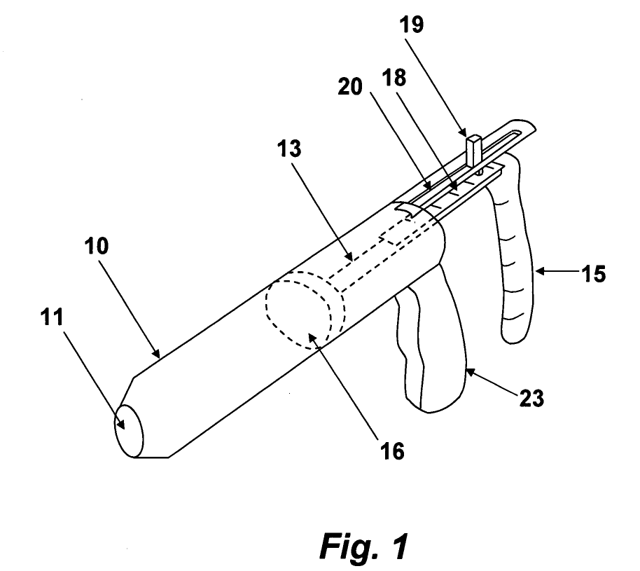

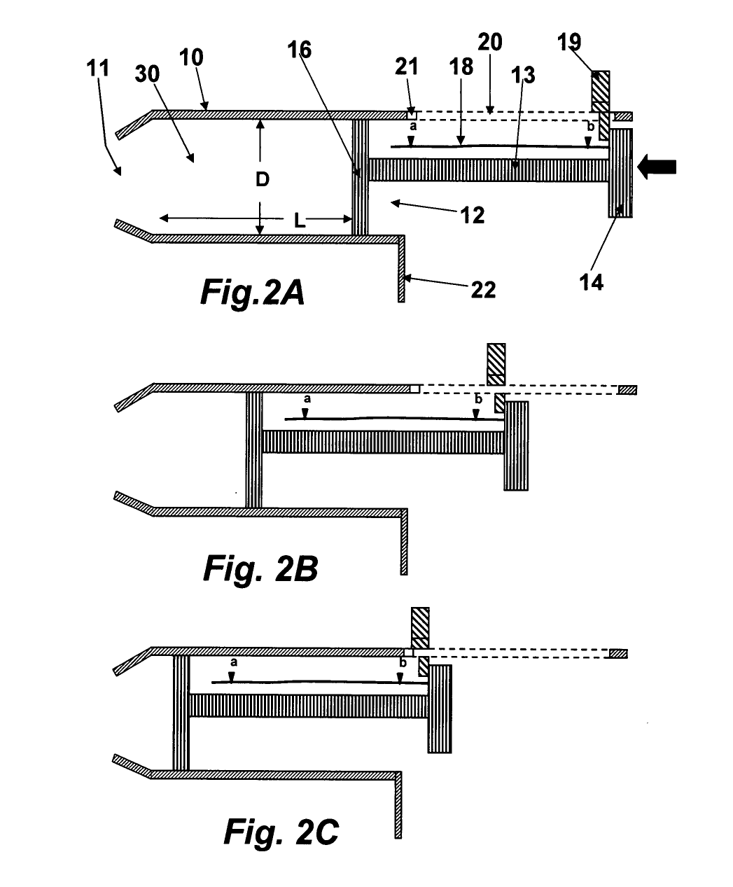

The present invention is directed to the problem of determining, without repeated calculation by the operator, the volume of a non-solid composition remaining in the barrel of a syringe after discharge of a dose of the composition. With reference now to FIGS. 1-4D, the present invention, as illustrated in FIG. 1 and shown in cross-section in FIGS. 2A-2C, provides a syringe barrel 10 having a distal discharge end 11 and a proximal plunger receiving end 12 and wherein the internal length ‘L’ and diameter ‘D’ of the barrel define an internal reservoir 30 having a volume sufficient to contain a desired dosage of a pharmaceutical composition.

The distal discharge end 11 may be configured for insertion into an orifice, such as the mouth, anus, ear channel, nostril, vagina, urethra or milk duct, of an animal. The dimensions of the distal discharge end 11 may be configured as to be acceptable to any animal to which it is desired to deliver a non-solid composition, including, but not limited to, a horse, a bovine, a sheep, a goat, a cat, a dog and the like. The distal discharge end 11 may also be configured to receive and retain a hypodermic or syringe needle, or a disposable tip that may, for example, be replaced between delivering doses of a composition to different animals. The dosage syringe of the invention further comprises a plunger 13 movably disposed within the barrel 10 and having a length greater than the internal length ‘L’ of the barrel 10.

The proximal end 14 of the plunger 13 may be configured to allow an operator to apply a positive pressure to the plunger 13. This positive pressure is then transferred to non-solid contents in the internal reservoir 30 of the barrel 10, which are then forced from the distal discharge end 11 and into the animal.

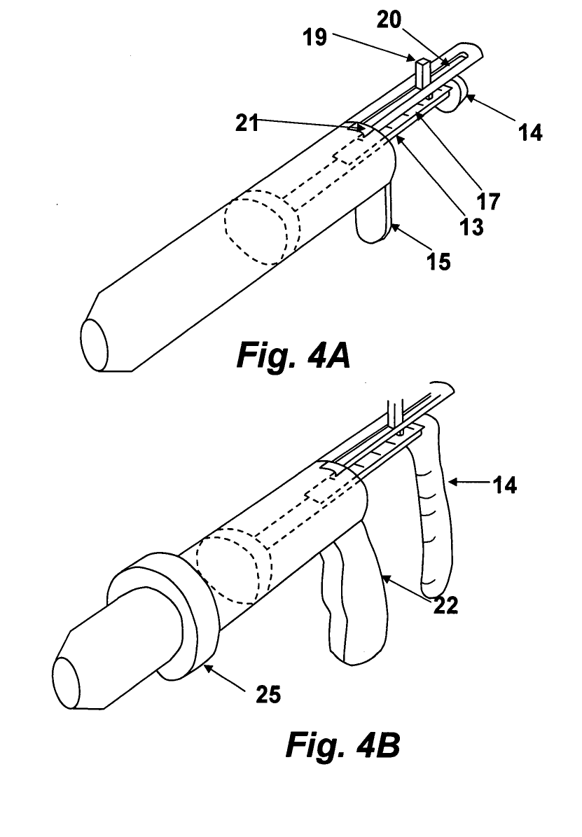

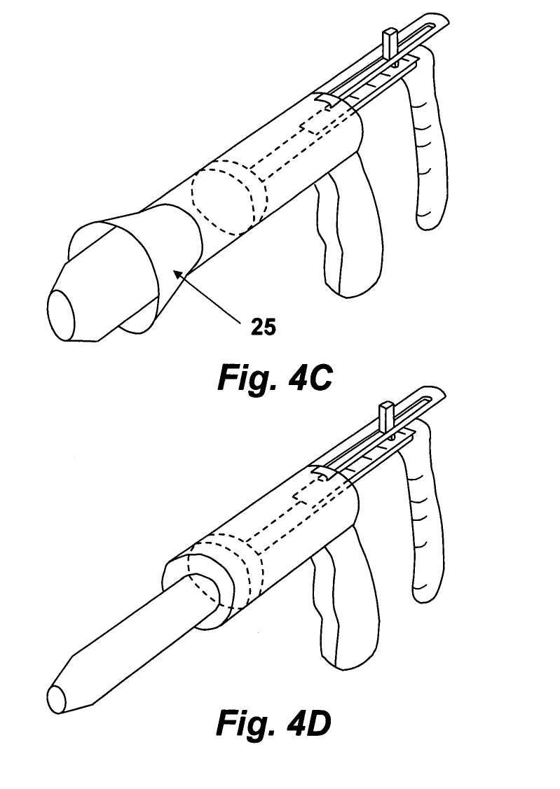

It is contemplated that the proximal end 14 of the plunger 13 may be a variety of forms such as, but not limited to, a thumb pad or a finger pad. In one embodiment of the invention, the proximal end 14 of plunger 13 is configured as an elongated handle 15 as shown in FIGS. 1 and 4B, whereby the pressure may be exerted by applying the palm of the operator. It is further contemplated that the barrel 10 may also comprise a protuberance 22, as shown in FIGS. 2A-2C and 4A, around which a finger or fingers of an operator may be partially wrapped to provide a resistance force against the pressure applied to the plunger 13, thereby facilitating depression of the plunger 13 into the barrel 10. The protuberance 22 may optionally be configured as a grip, as shown, for example in FIGS. 1, 4A and 4B, for the ergonomically placement of the operator's fingers when depressing the plunger 13. In one embodiment of the syringe of the invention, protuberance 22 and the proximal end 14 of plunger 13 are configured as elongated grips, as shown in FIG. 4B, that may be squeezed together by the operator to depress the plunger 13 into the barrel 10.

The plunger 13 also has a distal end having a seal means 16 disposed thereon and across the contents of the barrel 10 to prevent the contents from leaking towards the proximal end 14 of the plunger 13, and to transfer the positive pressure from the plunger 13 to the contents of the barrel 10 for discharging the contents through the distal discharge end 11.

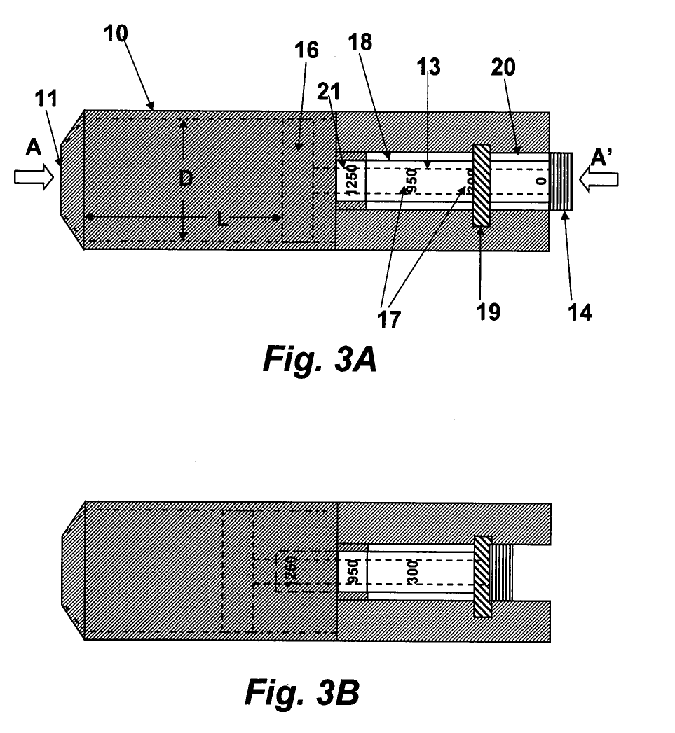

The plunger 13 further comprises a dose volume scale 17 disposed along the longitudinal axis of the plunger 13 and orientated such that the numeric values increase in the direction from the proximal end of the plunger towards the distal end 16, as shown, for example, in FIGS. 2A and 2B. The dose volume scale 17 may be printed, engraved or otherwise directly marked on the surface of the plunger 13. Alternatively, the scale may be a strip 18 of such as metal or plastic that is attached along its entire length to the plunger 13, or more loosely by attaching the proximal end, bearing the lowest indicated volume, to the proximal end 14 of the plunger 13, as shown, for example, in FIGS. 2A-3B. The dose volume scale 17 may be in any desired units such as, but not limited to lbs or kg of the recipient animal, volume of composition (ml's, cc's etc), units of a pharmaceutical agent and the like. Preferably, but not necessarily, only one scale 17 is provided to avoid misreading of the numeric values or units and delivering of an incorrect dose.

The dosage syringe of the present invention also comprises an adjustable dose selector 19 disposed at the proximal plunger receiving end 12 of the barrel 10 and configured that it may engage the proximal end 14 of the plunger 13 to prevent movement of the plunger 13 towards the distal discharge end 11 of the barrel 10 that would otherwise deliver a greater volume of the composition than is desired. In one embodiment of the invention, the dose selector 19 is slidably disposed within a slot 20 of the barrel 10 and parallel to the longitudinal axis of the plunger 13, the slot 20 overlaying the dose volume scale 17. Thus, the dose selector 19 may be moved to a volume mark of the scale 17 corresponding to a desired dose to be delivered. The plunger 13 may then be depressed until it encounters the dose selector 19, whereupon further depression is resisted, as shown in FIGS. 2A-2C. In various embodiments of the invention, as shown in FIGS. 1-4B, the dosage syringe comprises an extension 24 to the proximal plunger receiving end 12 of the barrel 10 and having the adjustable dose selector 19 and slot 20 disposed thereon.

Barrel 10 of the dose syringe of the invention may further comprise an indicator means 21 to indicate, from the dose volume scale, the amount of non-solid composition in the internal reservoir. In one embodiment of the syringe of the invention, the indicator means 21 may be an opening in the barrel 10 and positioned over the longitudinal axis of the plunger 13 and directly above the dose volume scale 17, such that when the plunger 13 is in a fully retracted position the window shows the volume of the full capacity of the syringe barrel 10. For example, as shown in FIG. 3A, when the plunger 13 is in the fully retracted position, the maximum capacity of the barrel 10 for the composition is 1250 units. When the plunger 13 has been depressed to deliver a 300 unit volume as shown, for example, in FIG. 3B, the dose scale 17 has also moved towards the distal discharge end 11 of the barrel 10 in unison with the plunger 13 (since it is attached to or on plunger 13) and indicates in the window 21 the remaining volume of composition, namely 1250 less 300, or 950 units. Accordingly, the remaining volume of composition is immediately presented without calculation by the syringe operator.

It is also contemplated that the indicator means 21 may be any configuration on the barrel 10 of the syringe such that, when the when the plunger 13 is in a fully retracted position, the indicator means 21 shows the volume of the full capacity of the syringe barrel 10. For example, but not in the limiting sense, a window in barrel 10 may be a vacant hole or opening, or filled with a transparent material, that allows the underlying scale 17 to be visible. In another embodiment, the distal end of the slot 20 may be located in such a position, as shown in FIG. 4B, that the distal numeric value showing is that of the volume of the contents remaining in the internal reservoir of the barrel 10.

In the various embodiments of the invention, the dosage syringe may also further comprise at least one protuberance 25 as shown, for example, in FIGS. 4B-4D, configured to limit entry of the barrel into the orifice of an animal, thereby both accurately placing the dispensed composition in the animal's orifice and avoiding injury to the animal. The protuberance may have a variety of forms including, but not limited to, a total (as shown in FIG. 4B), or partial annular form, an inverted cone (FIG. 4C) or a thickening of the barrel 10 relative to the diameter of the distal discharge end 11 inserted into an animal's orifice (FIG. 4D), and the like. Within the scope of the embodiments as shown in FIG. 4D may be syringes wherein the narrower region of the barrel is an attachable and/or disposable tip that may be changed between doses.

The present invention further provides kits comprising a syringe according to the present invention and instructions as to its mode of operation. The kits may further comprise at least one container enclosing a non-solid composition to be delivered to an animal. Any composition, preferably, but not limited to, a veterinary or pharmaceutical preparation may be included in the kits. When aspirated into the barrel 10 of the syringe by withdrawal of the plunger 13, the composition should be a fluid, either a solution or suspension of free-flowing particles, a preparation having high viscosity or a gel or paste capable of being discharged from the syringe upon depression of the plunger 13. The kits of the invention may also comprise a dosage syringe of the invention pre-filled with the desired composition, the flow properties of which also allowing discharge from the syringe.

One aspect of the present invention provides a dosage syringe comprising: a syringe barrel having a distal discharge end and a proximal plunger receiving end and wherein the internal length and diameter of the barrel define an internal reservoir having a sufficient volume to contain a desired dosage of a non-solid composition; a plunger movably disposed within the barrel and having a length greater than the internal length of the barrel, and having a proximal end configured to transfer an operator hand pressure to the plunger, a distal end having a seal means disposed across the contents of the barrel to prevent the contents from leaking and to transfer the hand pressure to the contents of the barrel for discharging the contents through the distal discharge end, and a dose volume scale disposed along the longitudinal axis of the plunger and orientated such that the numeric value increases in a direction from the proximal end of the plunger; an adjustable dose selector disposed at the proximal plunger receiving end of the barrel and configured to engage the proximal end of the plunger when the plunger is depressed, wherein the dose selector cooperates with the dose volume scale of the plunger to indicate a desired dose of a non-solid composition to be delivered by the syringe; and an indicator means disposed on the barrel to indicate on the dose volume scale the amount of the non-solid composition in the internal reservoir.

In the various embodiments of this aspect of the invention, the discharge end of the barrel may be configured for insertion into an orifice of an animal.

One embodiment according to the present invention further comprises a gripping means for securing the syringe during operation.

In another embodiment of the invention, the dosage syringe further comprises an extension to the proximal plunger receiving end of the barrel and having the adjustable dose selector disposed thereon.

In the various embodiments of the invention, the dosage syringe may further comprise at least one protuberance configured to limit the entry of the barrel into the mouth of an animal.

In the embodiments of the invention, the dose volume scale may be engraved, printed or attached along the longitudinal axis of the plunger.

In one embodiment of the invention, the dose volume scale is a linear strip and attached by a proximal end to the proximal end of the plunger and the distal end of the strip is movable disposed along the longitudinal axis of the barrel of the syringe.

In the various embodiments of the dosage syringe according to the invention, the indicator means disposed on the barrel to indicate on the amount of the pharmaceutical composition in the internal reservoir is selected from a window, an indicator mark or an elongated cutout from the barrel.

In one embodiment of the invention, the indicator means is a window.

The present invention also encompasses a dosage syringe comprising: a syringe barrel having a distal discharge end and a proximal plunger receiving end and wherein the internal length and diameter of the barrel define an internal reservoir having a sufficient volume to contain a desired dosage of a non-solid composition; plunger movably disposed within the barrel and having a length greater than the internal length of the barrel, and having a proximal end configured to transfer an operator hand pressure to the plunger, a distal end having a seal means disposed across the contents of the barrel to prevent the contents from leaking and to transfer the hand pressure to the contents of the barrel for discharging the contents through the distal discharge end, and a dose volume scale disposed along the longitudinal axis of the plunger and orientated such that the numeric value increases in a direction from the proximal end of the plunger, wherein the dose volume scale is engraved, printed, attached along the longitudinal axis of the plunger, or is a linear strip attached by a proximal end to the proximal end of the plunger and the distal end of the strip is movable disposed along the longitudinal axis of the barrel of the syringe; an adjustable dose selector disposed at the proximal plunger receiving end of the barrel and configured to engage the proximal end of the plunger when the plunger is depressed, wherein the dose selector cooperates with the dose volume scale of the plunger to indicate a desired dose of a non-solid composition to be delivered by the syringe; an indicator means disposed on the barrel to indicate on the dose volume scale the amount of the pharmaceutical composition in the internal reservoir; a gripping means for securing the syringe during operation; an extension to the proximal plunger receiving end of the barrel and having the adjustable dose selector disposed thereon; and at least one protuberance configured to limit the entry of the barrel into the mouth of an animal.

In the embodiments of this aspect of the invention, the indicator means disposed on the barrel to indicate on the amount of the pharmaceutical composition in the internal reservoir is selected from a window, an indicator mark or an elongated cutout from the barrel.

In one embodiment of this aspect of the invention, the indicator means disposed on the barrel to indicate on the amount of the pharmaceutical composition in the internal reservoir is a window.

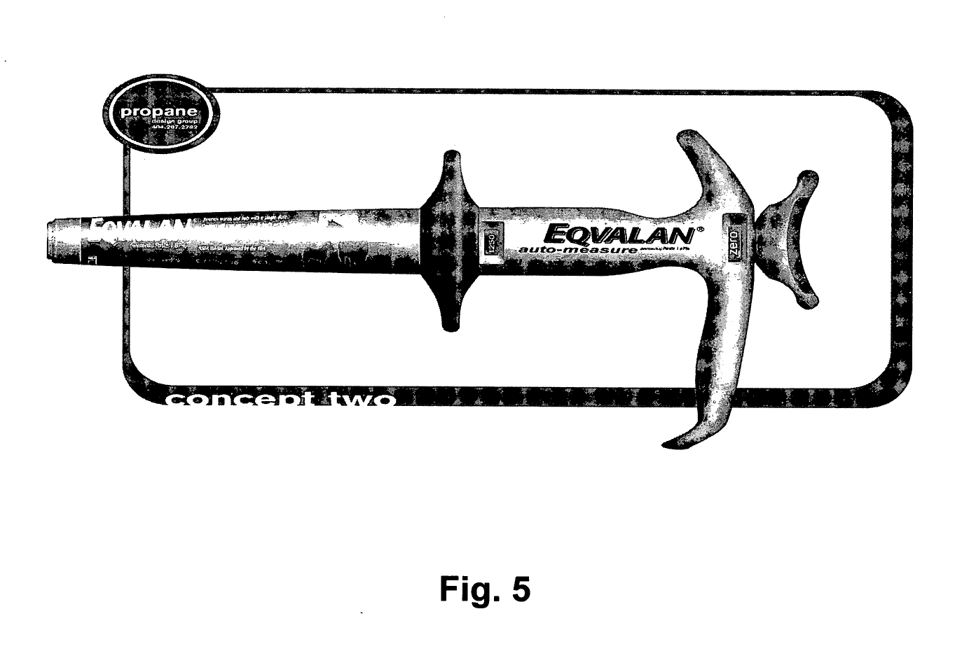

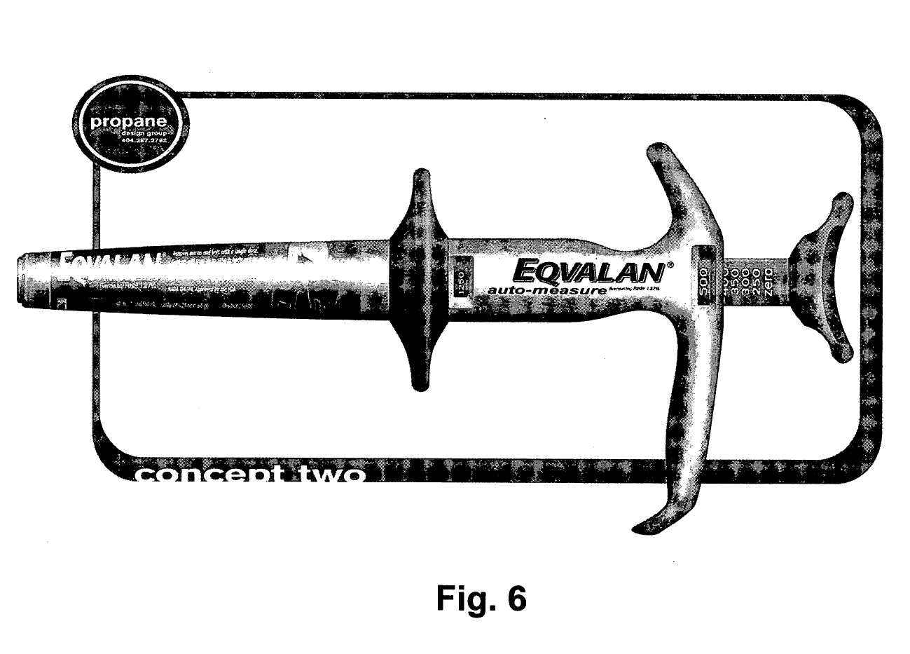

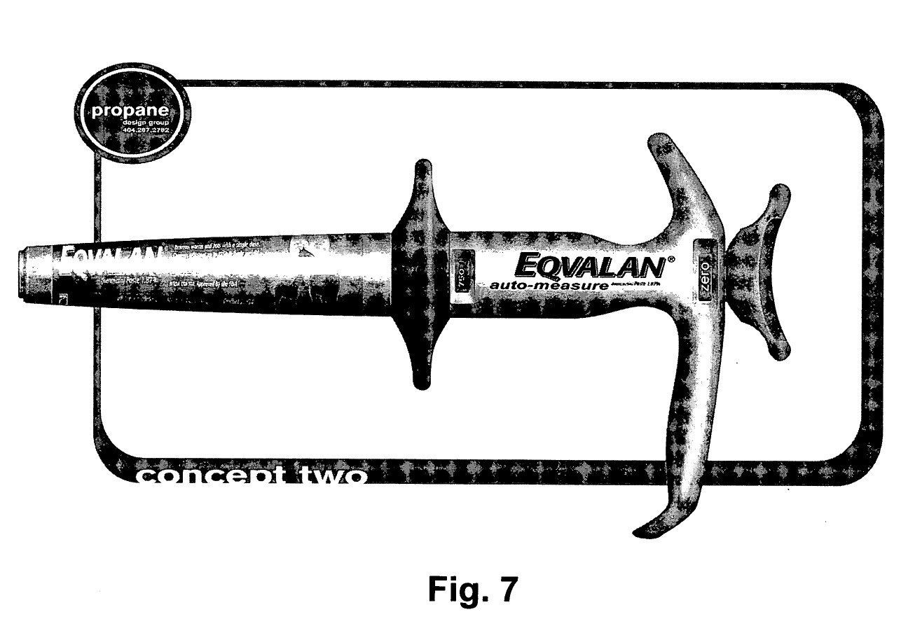

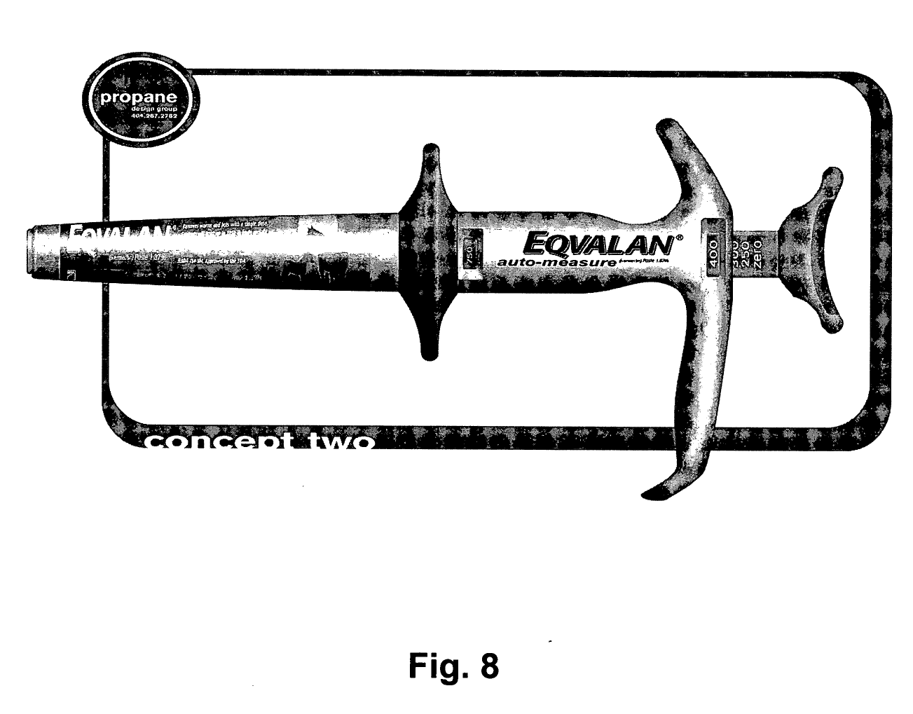

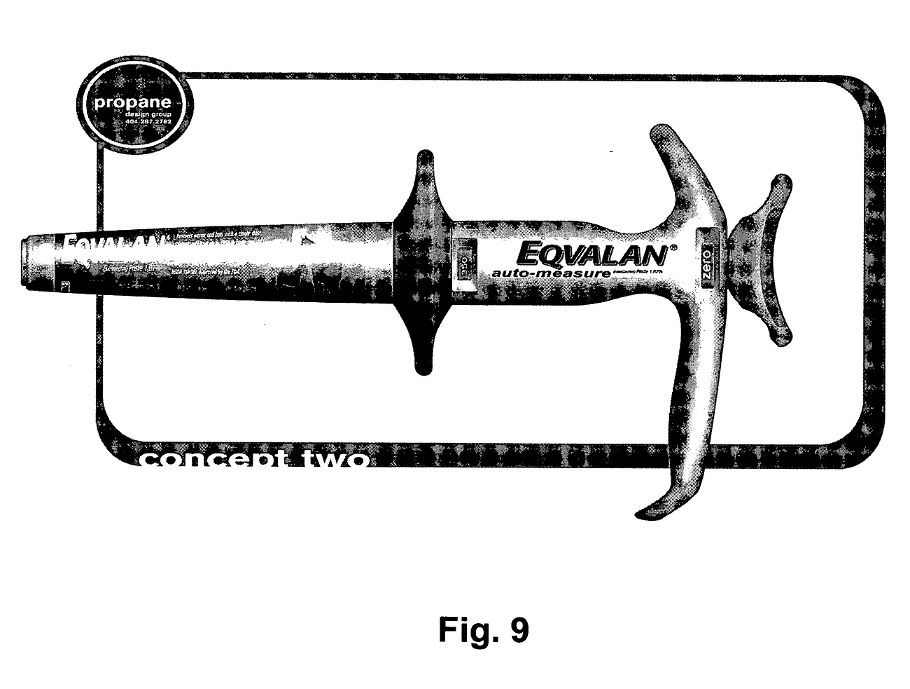

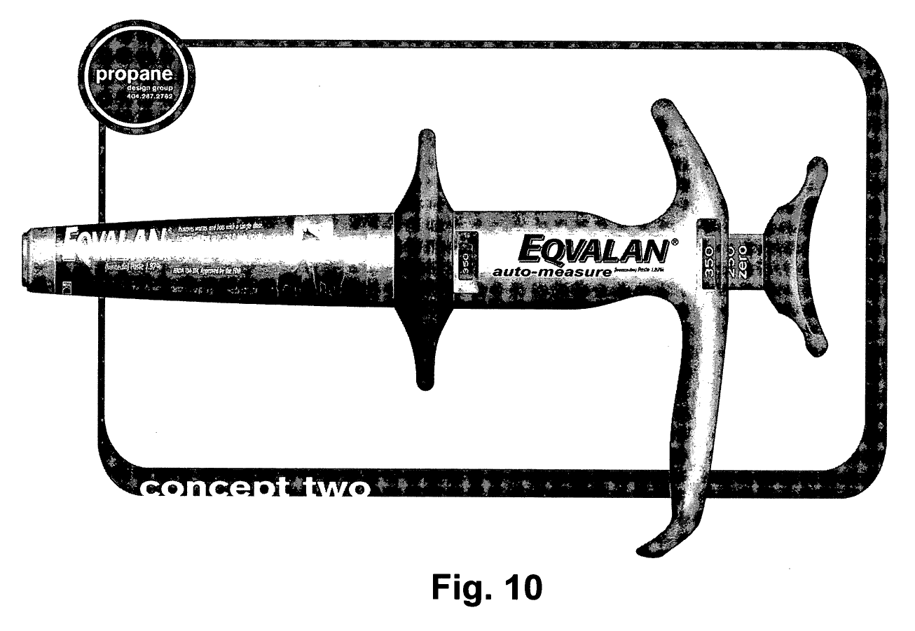

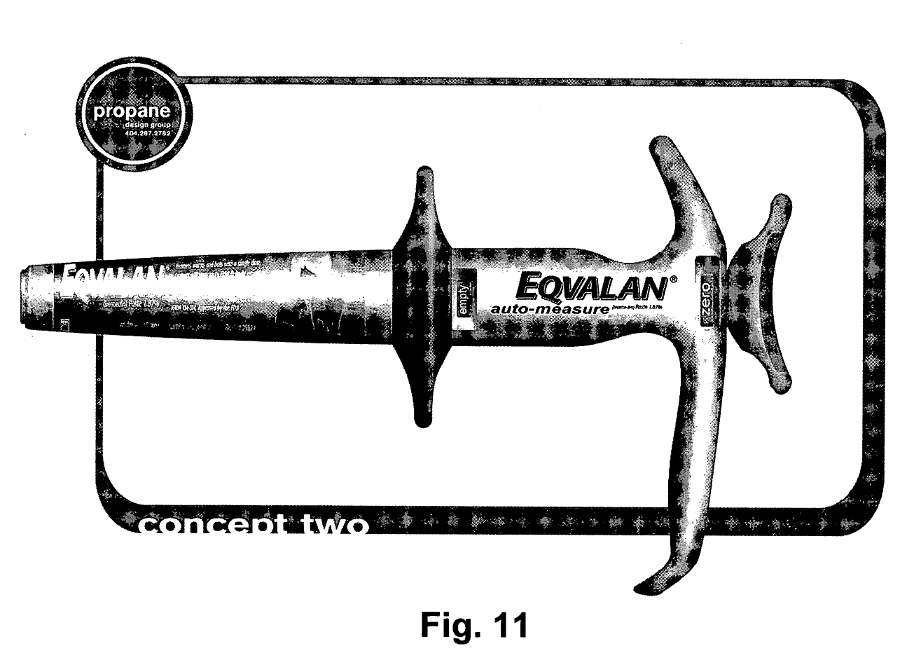

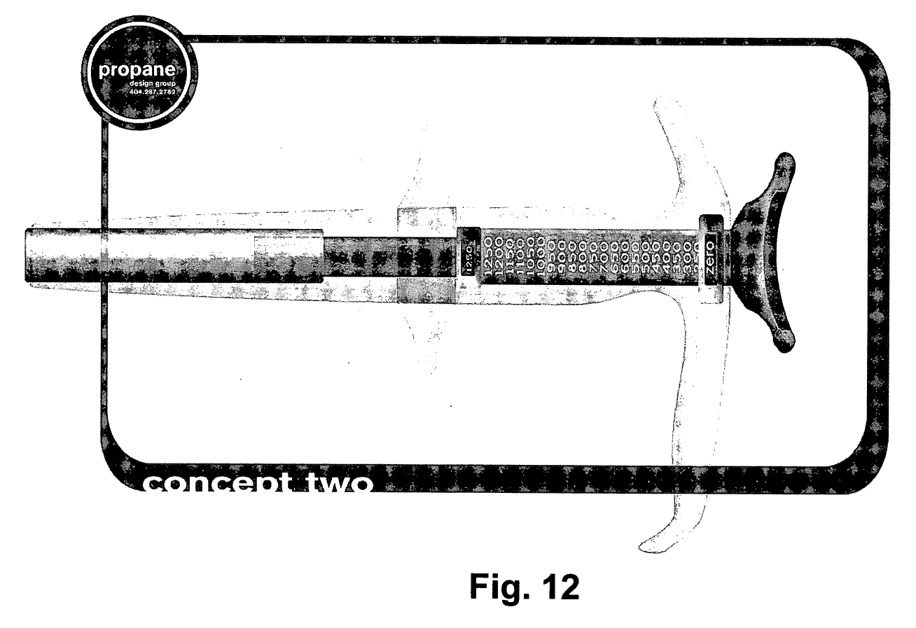

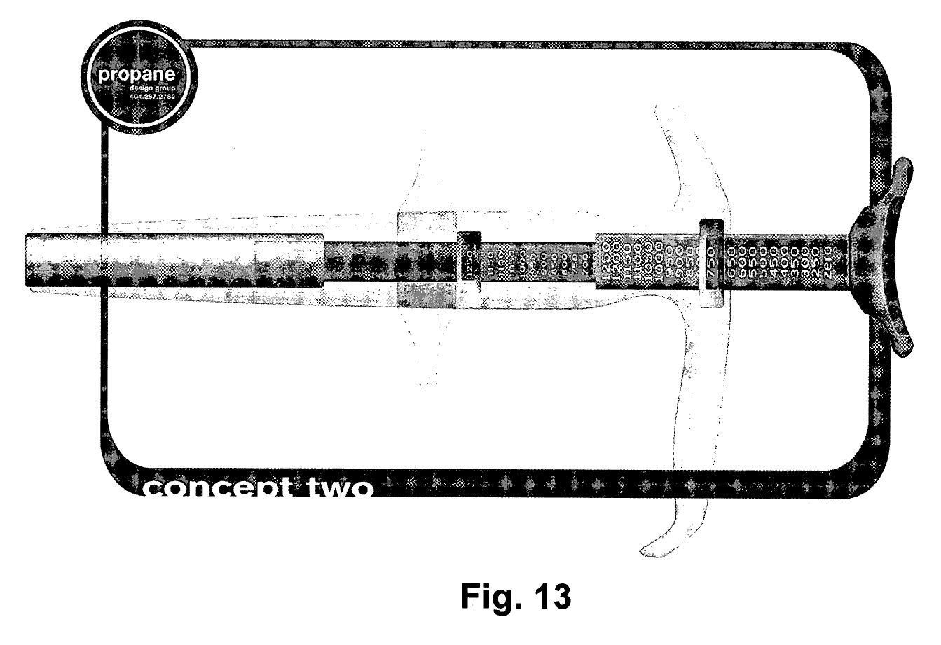

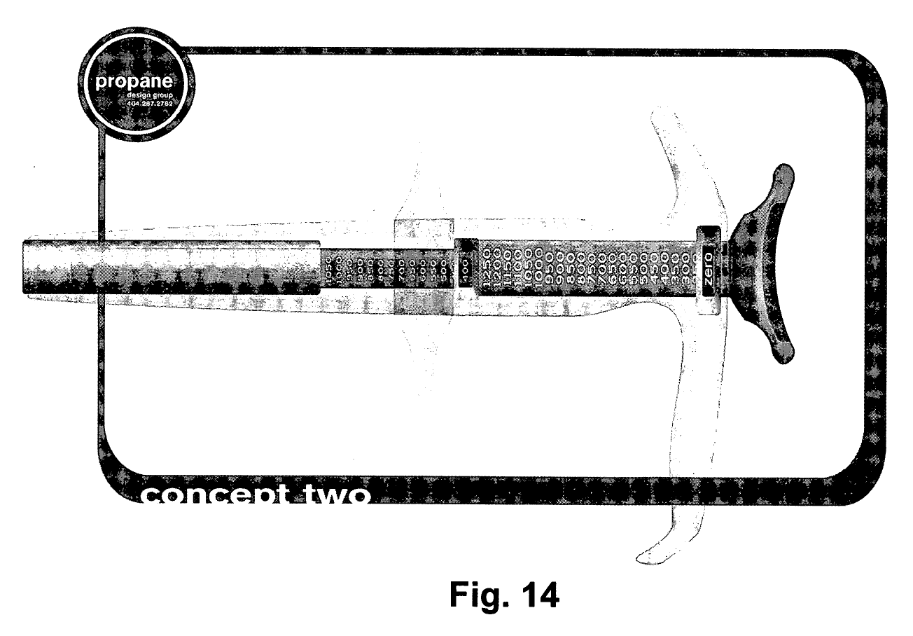

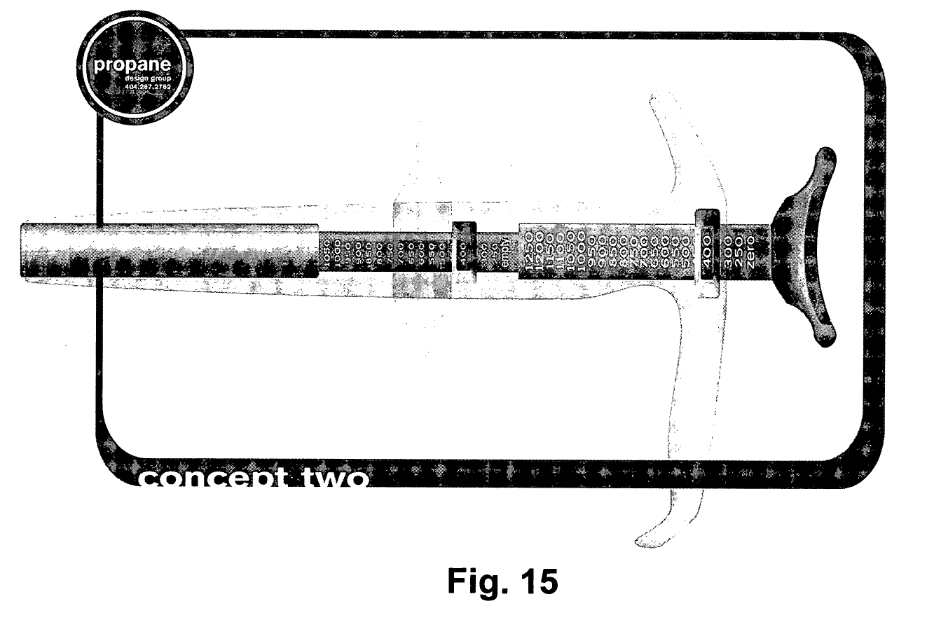

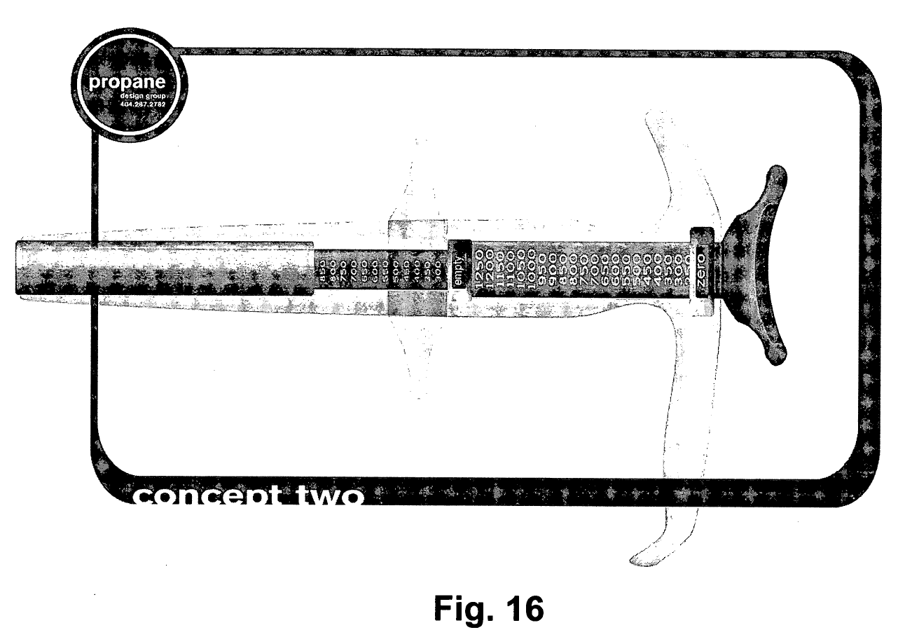

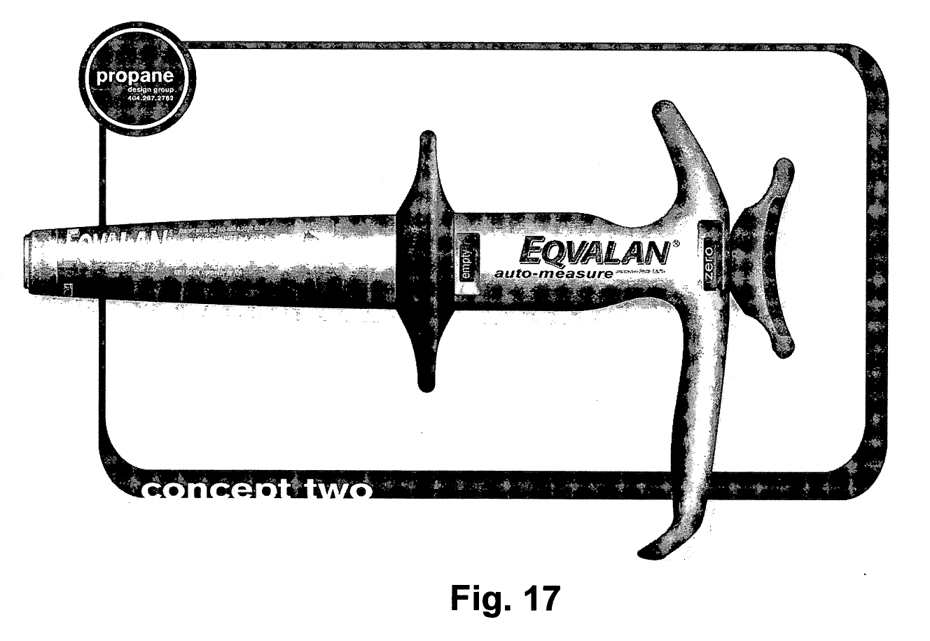

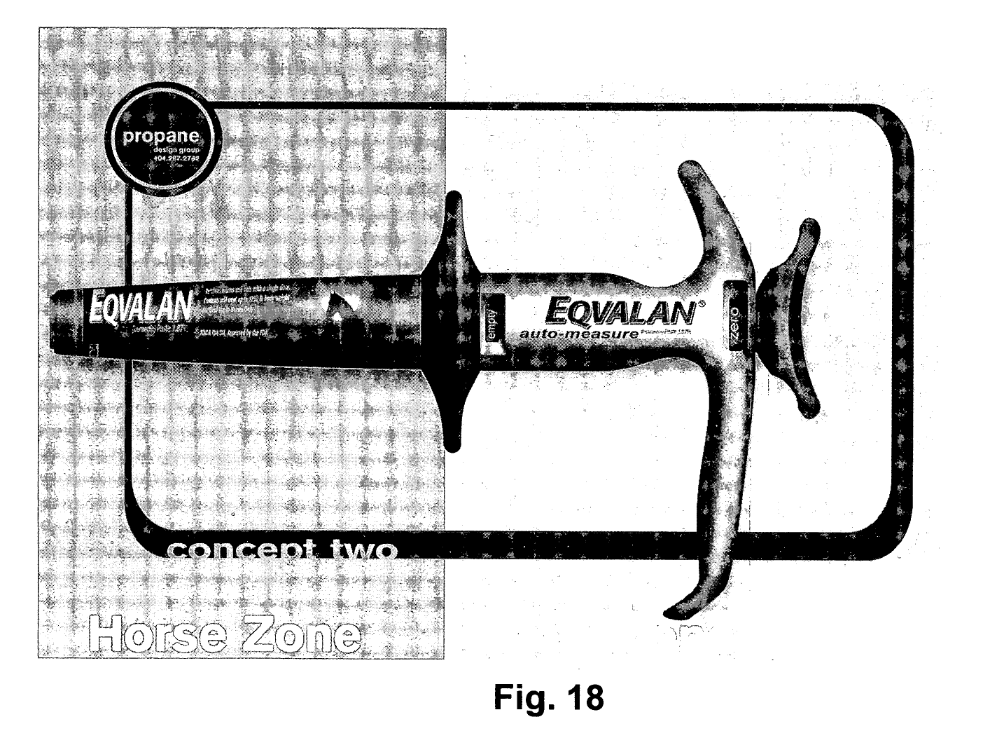

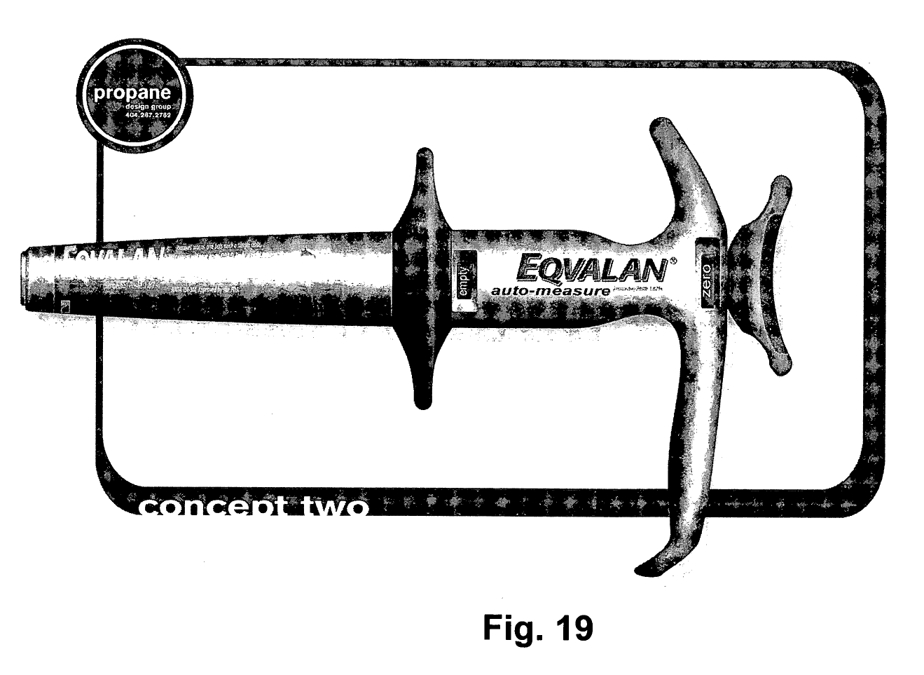

The invention further encompasses a dosage syringe wherein a second volume scale is disposed along the longitudinal axis of the plunger distal to the dose volume scale and in the reverse orientation to said dose scale such that the highest numeric value is at the distal end of the scale as shown, for example, in FIGS. 5-19. In this aspect of the invention, an indicator means may be located over the second volume scale to indicate the volume of the composition remaining in the barrel of the syringe. As the plunger is depressed into the barrel, the volume indicated from the second scale will decrease accordingly, as shown in FIGS. 5-19.

Another aspect of the invention provides a kit comprising a dosage syringe according to the present invention, and instructions for the use thereof to deliver a non-solid composition to an animal.

In one embodiment of this aspect of the present invention, the kit further comprises a container enclosing a non-solid composition for delivery to an animal, wherein the non-solid composition may be a fluid or a paste.

In another embodiment of the kits of the present invention, the dosage syringe is pre-filled with a non-solid composition for delivery to an animal.

Although methods and materials similar or equivalent to those described herein can be used in the practice or testing of the present invention, suitable methods and materials are described herein. All publications, patent applications, patents, and other references mentioned herein are incorporated by reference in their entirety. In addition, the materials, methods, and examples are illustrative only and are not intended to be limiting.

What is claimed is

1. A dosage syringe comprising:
(a) a syringe barrel having a distal discharge end and a proximal plunger receiving end and wherein the internal length and diameter of the barrel define an internal reservoir having a sufficient volume to contain a desired dosage of a pharmaceutical composition;
(b) a plunger movably disposed within the barrel and having a length greater than the internal length of the barrel, and having:
(i) a proximal end configured to transfer an operator hand pressure to the plunger;
(ii) a distal end having a seal means disposed across the contents of the barrel thereby preventing the contents from leaking from the internal reservoir and transferring pressure from the plunger to the contents of the barrel for discharging the contents through the distal discharge end; and
(iii) a dose volume scale disposed along the longitudinal axis of the plunger and orientated such that the numeric value increases in a direction from the proximal end of the plunger;
(c) an adjustable dose selector disposed at the proximal plunger receiving end of the barrel and configured to engage the proximal end of the plunger when the plunger is depressed, wherein the dose selector cooperates with the dose volume scale of the plunger to indicate a desired dose of a pharmaceutical composition to be delivered by the syringe; and
(d) an indicator means disposed on the barrel to indicate on the dose volume scale the amount of the pharmaceutical composition in the internal reservoir;

2. The dosage syringe according to claim 1, further comprising a gripping means for securing the syringe during operation.

3. The dosage syringe according to claim 1, further comprising an extension to the proximal plunger receiving end of the barrel and having the adjustable dose selector disposed thereon.

4. The dosage syringe according to claim 1, further comprising at least one protuberance configured to limit the entry of the barrel into the mouth of an animal.

5. The dosage syringe according to claim 1, wherein the dose volume scale is engraved, printed or attached along the longitudinal axis of the plunger.

6. The dosage syringe according to claim 1, wherein the dose volume scale is a linear strip and attached by a proximal end to the proximal end of the plunger and the distal end of the strip is movable disposed along the longitudinal axis of the barrel of the syringe.

7. The dosage syringe according to claim 1, wherein the indicator means is selected from a window, an indicator mark or an elongated cutout from the barrel.

8. The dosage syringe according to claim 1, wherein the indicator means is a window.

9. A dosage syringe comprising:
(a) a syringe barrel having a distal discharge end and a proximal plunger receiving end and wherein the internal length and diameter of the barrel define an internal reservoir having a sufficient volume to contain a desired dosage of a pharmaceutical composition;
(b) a plunger movably disposed within the barrel and having a length greater than the internal length of the barrel, and having:
(i) a proximal end configured to transfer an operator hand pressure to the plunger;
(ii) a distal end having a seal means disposed across the contents of the barrel to prevent the contents from leaking and to transfer the hand pressure to the contents of the barrel for discharging the contents through the distal discharge end; and
(iii) a dose volume scale disposed along the longitudinal axis of the plunger and orientated such that the numeric value increases in a direction from the proximal end of the plunger, wherein the dose volume scale is engraved, printed, attached along the longitudinal axis of the plunger, or is a linear strip attached by a proximal end to the proximal end of the plunger and the distal end of the strip is movable disposed along the longitudinal axis of the barrel of the syringe;
(c) an adjustable dose selector disposed at the proximal plunger receiving end of the barrel and configured to engage the proximal end of the plunger when the plunger is depressed, wherein the dose selector cooperates with the dose volume scale of the plunger to indicate a desired dose of a pharmaceutical composition to be delivered by the syringe;
(d) an indicator means disposed on the barrel to indicate on the dose volume scale the amount of the pharmaceutical composition in the internal reservoir;
(e) a gripping means for securing the syringe during operation;
(f) an extension to the proximal plunger receiving end of the barrel and having the adjustable dose selector disposed thereon; and
(g) at least one protuberance configured to limit the entry of the barrel into the mouth of an animal.

10. The dosage syringe according to claim 9, wherein the indicator means disposed on the barrel to indicate on the amount of the pharmaceutical composition in the internal reservoir is selected from a window, an indicator mark or an elongated cutout from the barrel.

11. The dosage syringe according to claim 10, wherein the indicator means disposed on the barrel to indicate on the amount of the pharmaceutical composition in the internal reservoir is a window.

12. The dosage syringe according to claim 1, wherein the discharge end of the barrel is configured for insertion into an orifice of an animal.

13. A kit comprising a dosage syringe according to claim 1, and instructions for the use thereof to deliver a non-solid composition to an animal.

14. The kit according to claim 13, further comprising a container enclosing a non-solid composition for delivery to an animal.

15. The kit according to claim 13, wherein the dosage syringe is pre-filled with a non-solid composition for delivery to an animal.

16. The kit according to claim 14, wherein the non-solid composition is a fluid or a paste.

17. The dosage syringe according to claim 1, further comprising a second volume scale distal to the dose volume scale and orientated in the reverse direction, wherein the two volume scales are disposed along the longitudinal axis of the plunger, and a volume indicator means, wherein the volume shown by the volume indicator means is the volume of composition remaining in the barrel of the dosage syringe.
